(19) United States
(12) Patent Application Publication
(10) Pub. No.: US 2006/0024374 A1
(43) Pub. Date: Feb. 2, 2006

(54) Pharmaceutical compositions suitable for the treatment of ophthalmic diseases

(76) Inventors: Maria Rosa Gasco, Torino (IT); Marco Fabrizio Saettone, Viareggio (IT); GIAN PAOLO ZARA, (US)

Correspondence Address: WILLIAM COLLARD;COLLARD & ROE, P.C., 1077 NORTHERN BOULEVARD, ROSLYN, NY, 11576, US

(21) Appl. No.: 10/533,512

(22) PCT Filed: Oct. 31, 2003
(86) PCT No.: PCT/EP03/12180

§ 371 Date: May 2, 2005

(30) Foreign Application Priority Data
Oct. 31, 2002 (IT) MI2002A002323

(51) Int. Cl. A61K 38/21; A61K 38/18; A61K 31/704; A61K 31/522; A61K 9/51

(52) U.S. Cl. 424/489; 514/34; 514/220; 514/263.38; 514/171; 514/12; 424/85.6

(57) ABSTRACT

Pharmaceutical compositions for the treatment of ophthalmic diseases, suitable for topical ocular administration and for systemic administration, comprising solid lipid nanoparticles (SLNs) with a mean diameter comprised between 50 and 400 nm wherein, within said nanoparticles, a pharmacologically active substance for the specific ophthalmic treatment is incorporated.

### KNOWN ART

Historically, the therapeutic treatment of the eye has been essentially directed towards the administration of drugs directly to the tissues and the fluids of the anterior segment of the eye.

Only recently has research been directed towards the administration of drugs intended for the posterior segment of the eye (uveal region, vitreous fluid, choroid and retina).

The eye is an isolated and highly protected organ.

In particular, the tight junctional complexes of the retinal pigmented epithelium and the retinal capillaries constitute the blood-retinal barrier for which the systemic administration of drugs does not succeed in reaching an adequate level within the posterior segment of the eye.

On the other hand, even with topical administration, only small amounts of drug reach the retina, as penetration through the external walls of the eye is very low. Nevertheless, there are numerous pathologies of the posterior segment of the eye which require pharmacological treatment such as for example bacterial or fungal endophthalmitis, viral retinitis, vitreoretinopathy, toxoplasmosis, uveitis, tumours, vascular diseases, diabetic retinopathy, age-related macular degeneration, glaucoma and others.

In order to overcome such difficulties, various methods of administration have been investigated.

Some authors have intravenously injected thermosensitive liposomes which have been lysed within the retinal vessels using microwave generated impulses (Khoobehi B. et al. Ophthalmology 1988 July, 95 (7): 950-5).

The injection of drugs into the vitreous fluid has also been described (Martidis A. et al. Ophthalmology 2002, 195 (5): 920-7).

However, frequently, this injection must be repeated and furthermore, can hold dangerous complications for the structures of the eye.

Finally, slow-release ocular implants have been proposed, such as for example Vitrasert®, an implant which is surgically inserted into the eye and releases ganciclovir over a period of six months. (Morley M G. et al Ophthalmologhy 1996; 103 (10): 1517).

From that reported above, it is clearly evident that only through very complicated, time consuming and expensive methods, is it possible to convey drugs to the posterior segment of the eye.

### SUMMARY

Now, pharmaceutical compositions suitable for the treatment of ophthalmic diseases have been found that allow to overcome the difficulties of the known art.

Said compositions comprise solid lipidic nanoparticles (SLNs) having mean diameter comprised between 50 and 400 nm and preferably comprised between 100 and 200 nm wherein, within said nanoparticles, a pharmacologically active substance for the specific ophthalmic treatment is incorporated.

Said compositions are prepared both in a form suitable for intravenous administration and a form suitable for topical ocular applications.

It has been found that the solid lipidic nanoparticles of the present invention are able to transport the drug to the vitreous fluid and to the retina, through the above mentioned administration routes, overcoming the difficulties of the known art.

### DETAILED DESCRIPTION OF THE INVENTION

The present invention refers to the use of solid lipidic nanoparticles (SLNs) for the preparation of pharmaceutical compositions suitable for the treatment of ophthalmic diseases.

A pharmacologically active substance for the specific ophthalmic treatment is incorporated within said nanoparticles.

The nanoparticles containing the pharmacologically active substance are prepared essentially according to the process described in European patent No 0526666 which comprises the following steps:
a) a molten lipid substance containing a drug or a complex thereof is mixed with a mixture comprising, and preferably consisting of water, a surfactant, a cosurfactant and optionally a counterion of the drug, pre-warmed to a 9 temperature at least equal to the melting temperature of said lipid substance, thus obtaining a microemulsion having a temperature at least equal to the melting temperature of said lipid substance;
b) the microemulsion obtained in step a) is dispersed in water or in an aqueous medium cooled to a temperature comprised of between 2 and 5° C., thus obtaining a dispersion of solid lipidic nanoparticles incorporating the drug;
c) the dispersion obtained in step b) is washed with water or with an aqueous medium by diafiltration with the practically total elimination of the surfactant and cosurfactant;
d) the dispersion obtained as in step c) is dried by lyophilisation or by spray drying or by evaporation, thus obtaining the solid lipidic nanoparticles (SLNs) with the incorporated drug.

The microemulsion of step a) can be sterilised by filtration using sterilising filters.

The dispersion obtained in step c) can be sterilised in an autoclave or by filtration using sterilising filters.

According to an alternative embodiment, the microemulsion obtained in step a) is added to a mixture comprising, and preferably consisting of, water, a surfactant, a cosurfactant and a lipid, warmed to a temperature at least equal to the melting temperature of the lipid and the mixture thus obtained is dispersed in water or in an aqueous medium cooled to a temperature comprised of between 2 and 5° C.

According to an additional alternative embodiment, at the end of step a) a substance suitable to sterically stabilise the lipidic nanoparticles is added.

The lipidic substances used in the process are selected from the group comprising:
triglycerides, particularly trilaurine, tricapriloin, tristearine, tripalmitine, capric/caprylic triglycerides (Mygliol®, Captex® and Labrafac®);
diglycerides, particularly dipalmitine and distearine;
monoglycerides, particularly glyceryl monostearate (Myvapex 600®) and glyceryl palmitostearate (Precirol®);
aliphatic alcohols, particularly cetylic alcohol and stearylic alcohol;
fatty acids having C10-C22 chains, decanoic acid, linoleic acid and polyalcohol esters thereof;
cholesterol and esters thereof, particularly cholesteryl hemisuccinate, cholesteryl butyrate and cholesteryl palmitate.

The surfactants are selected from the group comprising:
lecithins, as they are, such as Lipoid 75® and Epicuron 200®, phospholipids and hydrogenated forms thereof and synthetic and semi-synthetic derivatives thereof;
bile salts, particularly sodium glycocholate, sodium taurocholate and taurodeoxycholate;
Tween 20, Tween 40, Tween 80, Span 20, Span 40 and Span 60;
emulsifiers, particularly gelatin.

The cosurfactants are selected from the group comprising:
low molecular weight alcohols or glycols, particularly butanol, hexanol and hexadiol;
low molecular weight fatty acids, particularly butyric acid and octanoic acid;
phosphoric acid esters, benzylic alcohol and bile salts.

The substances suitable to sterically stabilise the lipidic nanoparticles are selected from dipalmitoyl phosphatidyl ethanolamine-PEG, diacyl phosphatidyl ethanolamine PEG (PEG M.W. 750-2000) and fatty acids pegylated with PEG-methylethers (PEG M.W. 750-2000).

The pharmacologically active substances suitable for the treatment of ophthalmic diseases according to the present invention can be both of the hydrophilic type and of the hydrophobic type and comprise antibiotics, antifungal agents, antiviral agents, antineoplastics, drugs for diabetic retinopathy, steroidal and non-steroidal anti-inflammatory agents, and antiglaucoma drugs. Preferably said pharmacologically active substances are selected from the group comprising: amphotericin, miconazole, ganciclovir, saquinavir, acyclovir, famciclovir, vidarabine, idoxuridine, β-interferon, paclitaxel, methotrexate, doxorubicin, angiopoietin 1, diclophenac, indomethacin, ketorolac, piroxicam, flurbiprofen, dexamethasone, triamcinolone, hydrocortisone, fluorometholone, rimexolone, timolol, betaxolol and acetazolamide.

The solid lipidic nanospheres (SLNs) of the present invention have a mean diameter comprised between 50 and 400 nm and preferably comprised between 100 and 200 nm and a polydispersion comprised between 0.06 and 0.30 and preferably comprised between 0.10 and 0.20.

Said SLNs have a pharmacologically active substance content comprised between 0.1 and 7.0%.

They are used for the preparation of pharmaceutical compositions for intravenous administration or for topical ocular administration.

The compositions for intravenous administration are prepared by dispersion of the SLNs in isotonic aqueous solutions in such quantities as to obtain a concentration of SLNs comprised between 10 and 250 mg/ml.

Preferably said aqueous solution is made isotonic by the addition of glycerol.

The compositions for topical ocular administration are prepared in the same manner with the further addition of 0.1-0.4% of a viscosizing substance, for example polyvinyl alcohol or hydroxypropyl cellulose, and contain 1.0 to 25% W/v SLNs.

The present invention also refers to a therapeutic method for the treatment of ophthalmic diseases comprising, and preferably consisting in, the intravenous or topical ocular administration of a therapeutically effective amount of a pharmaceutical composition as defined above.

The dosage for intravenous administration is of an amount of composition containing 0.01-5.0 milligrams of active substance per kilogram of body weight.

The dosage for topical ocular administration is of an amount of composition containing 0.01-5.0 mg of active substance per eye.

The compositions according to the present invention have important advantages compared to the known art with regard to both the simplicity of preparation and application and the efficacy of the active substance.

Indeed they allow the transport of the SLNs to the posterior segment of the eye following both systemic and topical ocular administration.

In any case, the blood-retinal barrier is easily overcome and the active substance incorporated within the SLNs reaches the vitreous fluid and the retina.

It shall be noted that said compositions allow the transport across the blood-retinal barrier even of active substances that are practically insoluble in an aqueous medium.

Finally, the compositions for intravenous administration can be constituted by sterically stabilised SLNs as already observed, with the advantage of minimising their uptake by macrophages.

For the purpose of illustration of the preparation process of the solid lipidic nanoparticles, of the product obtained and of the effects of its ophthalmic administration, the following examples are reported.

### EXAMPLE 1

### Preparation of the SLNs

200 mg of molten stearic acid at a temperature of 70° C. containing a 1:2 gentamicin-hexadecylphosphate (28.85 mg, equivalent to 12 mg of gentamicin) complex are added to a mixture constituted by filtered water (2 ml), Epikuron 200® (105 mg) and sodium taurocholate (285 mg) warmed to a temperature of 70° C.

The microemulsion obtained, having a temperature of 70° C., is dispersed in water in a volume ratio of 1/5 at a temperature of 2-3° C. by mechanical stirring obtaining a dispersion of solid lipidic nanoparticles (SLNs).

The dispersion obtained is washed twice with water for injection by diafiltration.

The SLNs have a mean diameter of 75 nm and a polydispersion of 0.2 and the lyophilised product has a gentamicin content of 3.3%.

### EXAMPLE 2

### Intravenous Administration

An isotonic aqueous dispersion has been prepared with the solid lipidic nanoparticles (SLNs) prepared according to example 1, having a concentration of SLNs corresponding to 6 mg/ml of gentamicin.

The dispersion has been injected into the marginal ear vein of three male New Zealand albino rabbits having weights of 2.8-3.5 kg. The injected dose of gentamicin has been 1.5 mg/kg.

The commercial composition Gentomil®, containing the same dose of gentamicin, has been injected as a control into other three rabbits having the same characteristics.

One hour after administration, the following results, which represent the mean values of the gentamicin concentrations in various ocular areas, have been obtained.

(a) Dispersion of SLNs:

concentration of gentamicin in the aqueous fluid:
right eye=300 ng/100 μl
left eye=326 ng/100 μl
concentration of gentamicin in the vitreous fluid:
right eye=499 ng/100 μl
left eye=531 ng/100 μl
concentration of gentamicin in the retina:
right eye=1225 ng/100 μl
left eye=1365 ng/100 μl
   (b) Gentomil® Composition
Concentration of gentamicin in the aqueous fluid:
right eye=50 ng/100 μl
left eye=56 ng/100 μl
Concentration of gentamicin in the vitreous fluid:
right eye=3.5 ng/100 μl
left eye=2.5 ng/100 μl
Concentration of gentamicin in the retina: non perceptible.

### EXAMPLE 3

### Intravenous Administration

Example 2 has been repeated with the difference that the dose injected has been 2 mg/kg.

Three hours after administration, the following results have been obtained.

(a) Dispersion of SLNs:

concentration of gentamicin in the aqueous fluid:
right eye=244 ng/100 μl
left eye=120 ng/100 μl
concentration of gentamicin in the vitreous fluid:
right eye=126 ng/100 μl
left eye=157 ng/100 μl
concentration of gentamicin in the retina:
right eye=99.5 ng/100 μl
left eye=84 mg/100 μl
   (b) Gentomil® Composition
Concentration of gentamicin in the aqueous fluid:
right eye=40 ng/100 μl
left eye=36 ng/100 μl
Concentration of gentamicin in the vitreous fluid: not perceptible
Concentration of gentamicin in the retina:
not perceptible

### EXAMPLE 4

### Topical Ocular Administration

An isotonic aqueous dispersion has been prepared with the solid lipid nanoparticles (SLNs) prepared according to example 1, having a concentration of SLNs corresponding to 2 mg/ml of gentamicin.

Polyvinyl alcohol (M.W. 20,000) has been added to the dispersion as a viscosizing agent, in an amount of 0.2% with respect to the dispersion.

Three rabbits having the characteristics described in example 2 have been used for the experiment.

The administration has been carried out by topically administering 50 μl of SLNs dispersion into the lower conjunctival sack of one eye of each rabbit.

As a control, the same dose of gentamicin has been administered in the same manner to other three rabbits having the same characteristics, by means of a commercial composition denominated Genticol®).

One hour after administration, the following results, which represent the mean values of the concentrations of gentamicin within the eye, have been obtained.

(a) Dispersion of SLNs:

Concentration of gentamicin in the aqueous fluid=10 μg/100 μl
concentration of gentamicin in the vitreous fluid=2.76 μg/100 μl
concentration of gentamicin in the retina=890 ng/100 μl
   (b) Genticol® Composition
Concentration of gentamicin in the aqueous fluid=5 g/100 μl
Concentration of gentamicin in the vitreous fluid: not perceptible
Concentration of gentamicin in the retina: not perceptible.

### EXAMPLE 5

### Topical Administration

Example 4 has been repeated with the difference that a dose of 200 μl has been administered.

One hour after administration the following results have been obtained which represent the mean values of the concentrations of gentamicin in the eye.

(a) Dispersion of SLNs:

concentration of gentamicin in the aqueous fluid=35 μg/100 μl
concentration of gentamicin in the vitreous fluid=7.84 μg/100 μl
concentration of gentamicin in the retina=5.4 μg/100 μl
   (b) Genticol® Composition
concentration of gentamicin in the aqueous fluid=16 μg/100 μl
concentration of gentamicin in the vitreous fluid=trace
concentration of gentamicin in the retina=trace.

1. (canceled)

2. The therapeutic method according to claim 11, wherein said SLNs have a mean diameter comprised between 50 and 400 nm, and a polydispersion comprised of between 0.06 and 0.30.

3. The therapeutic method according to claim 11, said SLNs have an average diameter comprised between 100 and 200 nm and a polydispersion comprised of between 0.10 and 0.20.

4. The therapeutic method according to claim 11, wherein said SLNs have a pharmacologically active substance content comprised of between 0.1 and 7.0%.

5. (canceled)

6. (canceled)

7. The therapeutic method according to claim 1, wherein said pharmacologically active substance is selected from the group comprising: amphotericin, miconazole, ganciclovir, saquinavir, acyclovir, famciclovir, vidarabine, idoxuridine, β-interferon, paclitaxel, methotrexate, doxorubicin, angiopoietin 1, diclophenac, indomethacin, ketorolac, piroxicam, flurbiprofen, dexamethasone, triamcinolone, hydrocortisone, fluorometholone, rimexolone, timolol, betaxolol and acetazolamide.

8. The therapeutic method according to claim 11, wherein said SLNs are prepared by a process wherein:
a) a molten lipid substance containing a drug or its complex is mixed with a mixture comprising water, a surfactant, a cosurfactant and optionally a counterion of the drug, pre-warmed to a temperature at least equal to the melting temperature of said lipid substance, thus obtaining a microemulsion having a temperature at least equal to the melting temperature of said lipid substance;
b) the microemulsion obtained in step a) is dispersed in water or in an aqueous medium cooled to a temperature comprised between 2 and 5° C., thus obtaining a dispersion of solid lipidic nanoparticles incorporating the drug;
c) the dispersion obtained in step b) is washed with water or with an aqueous medium by diafiltration with the practically total elimination of the surfactant and the consurfactant;
d) the dispersion obtained in step c) is dried by lyophilisation or by spray drying or by evaporation, thus obtaining the solid lipid nanoparticles (SLNs) with the drug incorporated.

9. The therapeutic method according to claim 8, wherein the microemulsion obtained in step a) is added to a mixture comprising water, a surfactant, a consurfactant and a lipid warmed to a temperature at least equal to the melting temperature of the lipid and the mixture thus obtained is dispersed in water or in an aqueous medium cooled to a temperature comprised of between 2 and 5° C.

10. The therapeutic method according to claim 8, wherein at the end of step a) a substance suitable for stabilising the SLNs is added selected from the group comprising dipalmitoyl phosphatidyl ethanolamine-PEG, diacyl phosphatidyl ethanolamine-PEG (PEG M. W. 750-2000) and fatty acids pegylated with PEG-methylethers (PEG M.W. 750-2000).

11. A therapeutic method for the treatment of ophthalmic diseases comprising the intravenous or topical ocular administration of a therapeutically effective amount of a pharmaceutical composition comprising solid lipidic nanoparticles containing a pharmacologically active substance suitable for the treatment of said ophthalmic diseases.

12. The therapeutic method according to claim 11, wherein the dosage for intravenous administration is an amount of said composition containing to 0.01-5.0 milligrams of active substance per kilogram of body weight.

13. The therapeutic method according claim 11, wherein the dosage for topical ocular administration is an amount of said composition containing to 0.01-5.0 milligrams of active substance for each eye.

14. A pharmaceutical composition suitable for the treatment of ophthalmic diseases by intravenous or topical ocular administration, consisting essentially of an isotonic aqueous dispersion of solid lipid nanoparticles (SLNS) having a mean diameter comprised between 50 and 400 nm and polydispersion comprised between 50 and 400 nm and polydispersion comprised between 0.06 and 0.30, a pharmacologically active substance for the treatment of said diseases being incorporated within said SLNs.

15. The parmaceutical composition according to claim 14, wherein said aqueous dispersion contains a viscosizing substance.

16. The composition according to claim 14, wherein said SLNs have a mean diameter comprised between 100 and 200 nm and polydispersion comprised between 0.10 and 0.20.

17. The composition according to claim 14, wherein the intravenous administration, said isotonic aqueous dispersion has a concentration of SLNs comprised of between 10 and 250 mg/ml.

18. The composition according to claim 14, wherein for the topical ocular administration, said isonic aqueous dispersion has a concentration of SLNs comprised between 1 and 25% w/v and contains from 0.1 to 0.4% of a viscosizing substance.

19. The composition according to claim 14, wherein said SLNs have a pharmacologically active substance content comprised between 0.1 and 7.0%:

20. The composition according to claim 14, wherein said pharmacologically active substance is selected from the group comprising: amphotericin, miconazole, ganciclovir, saquinavir, acyclovir, famciclovir, vidarabine, idoxuridine, β-interferon, paclitaxel, methotrexate, doxorubicin, angiopoietin 1, diclophenac, indomethacin, ketorolac, piroxicam, flurbiprofen, dexamethasone, triamcinolone, hydrocortisone, fluorometholone, rimexolone, timolol, betaxolol e acetazolamide.

21. Compositions according to claim 14, wherein the lipid of said SLNs is selected from the group comprising trilaurine, tricapriloin,
